(19) United States
(12) Patent Application Publication
(10) Pub. No.: US 2005/0182477 A1
(43) Pub. Date: Aug. 18, 2005

(54) Intraluminal stent and graft

(76) Inventor: Geoffrey H. White, Birchgrove (AU)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/499,016

(22) PCT Filed: Dec. 20, 2002
(86) PCT No.: PCT/AU02/01757

(30) Foreign Application Priority Data
Dec. 20, 2001 (AU) PR9693
Dec. 20, 2001 (AU) PR9694

(51) Int. Cl. A61F 2/06; A61F 2/04

(52) U.S. Cl. 623/1.15; 623/23.7

(57) ABSTRACT

An intraluminal stent which includes a tubular body made up of a plurality of unit cells. Each unit cells has a first end portion adjacent a first end and a second end portion adjacent a second end wherein the first end portion is of a greater dimension than the dimension of the second end portion. An intraluminal graft which includes a tubular body reinforced by a plurality of unit cells. Each unit cell has a first end portion adjacent a first end and a second end portion adjacent a second end wherein the first end portion is of a greater dimension that the dimension of the second end portion.

### FIELD OF THE INVENTION

The present invention relates to a graft and to a stent for use in the treatment of diseases of the vasculature and other vessels of a subject.

### BACKGROUND OF THE INVENTION

Diseases affecting the vasculature (or other vessels) are common and include atherosclerosis and aneurysmal disease.

Current medical practices employ both invasive and non-invasive procedures to treat diseases of the vasculature. In this regard, while many diseases may be medically treated, in severe cases, particularly in the case of aneurysmal disease or severe stenotic disease, surgical intervention may be required.

One means of treating aneurysmal disease is to bridge the diseased area with a graft. The graft is a hollow tubular structure which allows the flow of blood therethrough.

Conventional grafts may be inserted percutaneously through a distal and connecting vessel to that in which the graft is to be used. Upon release of the device from the catheter it may expand to a desired size, and may extend above and below the diseased section of vessel, thereby bridging that section.

To be effective in providing a stable bridge for the flow of blood through a diseased section of vessel, the graft must have good strength and flexibility while also having a good expansile ratio. This allows the graft to be packaged in a compressed form into a suitable introducer catheter while at the same time providing an expanded form of suitable diameter to engage the wall of a vessel in which it is placed.

Conventional grafts are typically made from a Dacron outer sheath which is reinforced by a circumferential series of wires. While typically quite flexible, such grafts may not have adequate strength to bridge a particular diseased section of vessel.

Atherosclerosis is characterised by a build up of plaque from cholesterol residues. The plaque build up subsequently thickens and hardens the vessel wall to create a stenosis. The resultant narrowing of the vessel has adverse effects on blood flow through the vessel.

As noted above, both invasive and non-invasive procedures may be employed to treat stenosis or other diseases of a vessel. While stenosis may be medically treated, in severe cases surgical intervention may be required. The latter includes both balloon angioplasty to break up the stenotic plaque and the delivery of an intraluminal stent to bridge the stenotic lesion and prevent re-stenosis.

While both procedures are commonly used, the incidence of re-stenosis in patients treated by balloon angioplasty is unacceptably high at an estimated 40% of cases. Bridging of the stenotic lesion with a stent significantly reduces the incidence of re-stenosis.

Conventional stents may be inserted percutaneously through a distal and connecting vessel to that in which the stent is to be used. For example, the device may be inserted through the femoral artery in a catheter, where the device is intended to be used in the treatment of a stenotic lesion. Upon release of the device from the catheter it may expand to a desirable size, and may extend above and below the lesion thereby bridging that lesion.

The first stents used clinically were the self expanding “Wallstents” which were made from a metallic mesh material. Subsequent designs included the Palmaz-Schatz slotted tube stents which were originally made from slotted stainless steel tubes comprising separate segments and the Wiktor stents which comprised a tube formed of a single strand of tantalum metal wound in a sinusoidal helix. However, each of the prior art stents have limitation with respect to flexibility, strength and expansile ratio.

The present invention aims to provide a graft and a stent both of which have features which address the limitations of the prior art.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

In a first aspect, the present invention consists in an intraluminal stent comprising a tubular body extending from a proximal end to a distal end, said tubular body being capable of expanding or being expanded from a radially compressed state to a radially expanded state, characterised in that the tubular body includes a plurality of unit cells, each unit cell having a first end portion adjacent a first end and a second end portion adjacent a second end and wherein the first end portion is of a greater dimension than the dimension of the second end portion.

In a second aspect, the present invention consists in an intraluminal stent comprising a tubular body extending from a proximal end to a distal end, said tubular body being capable of expanding or being expanded from a radially compressed state to a radially expanded state, characterised in that the tubular body includes a plurality of unit cells, each unit cell having a longitudinal axis and a transverse axis, wherein each unit cell is symmetrical about its longitudinal axis and asymmetrical about its transverse axis.

In a third aspect, the present invention consists in an intraluminal stent comprising a tubular body extending from a proximal end to a distal end, said tubular body being capable of expanding or being expanded from a radially compressed state to a radially expanded state, characterised in that the tubular body includes a plurality of unit cells, each unit cell having a first end portion adjacent a first end and a second end portion adjacent a second end, wherein the first end portion is of a greater dimension than the dimension of the second end portion and each unit cell has a longitudinal axis and a transverse axis, each cell being symmetrical about its longitudinal axis and asymmetrical about its transverse axis.

In a fourth aspect, the present invention consists in an intraluminal stent comprising a tubular body extending from a proximal end to a distal end, said tubular body being capable of expanding or being expanded from a radially compressed state to a radially expanded state, characterised in that the tubular body includes a plurality of unit cells, wherein each unit cell comprises a first end portion comprising a plurality of tapering regions and a second end portion comprising at least one tapering region.

In a fifth aspect, the present invention consists in an intraluminal graft comprising a tubular body which extends from a proximal end to a distal end and which is capable of expanding or being expanded from a radially compressed state to a radially expanded state, characterised in that said tubular body is circumferentially reinforced along at least part of its length by a plurality of unit cells, each unit cell having a first end portion adjacent a first end and a second end portion adjacent a second end, wherein the first end portion is of a greater dimension than the dimension of the second end portion.

In a sixth aspect, the present invention consists in an intraluminal graft comprising a tubular body which extends from a proximal end to a distal end and which is capable of expanding or being expanded from a radially compressed state to a radially expanded state, characterised in that said tubular body is circumferentially reinforced along at least part of its length by a plurality of unit cells, each unit cell having a longitudinal axis and a transverse axis, wherein each unit cell is symmetrical about its longitudinal axis and asymmetrical about its transverse axis.

In a seventh aspect, the present invention consists in an intraluminal graft comprising a tubular body which extends from a proximal end to a distal end and which is capable of expanding or being expanded from a radially compressed state to a radially expanded state, characterised in that said tubular body is circumferentially reinforced along at least part of its length by a plurality of unit cells, each unit cell having a first end portion adjacent a first end and a second end portion adjacent a second end, wherein the first end portion is of a greater dimension than the dimension of the second end portion and each unit cell has a longitudinal axis and a transverse axis, each cell being symmetrical about its longitudinal axis and asymmetrical about its transverse axis.

In an eighth aspect, the present invention consists in an intraluminal graft comprising a tubular body which extends from a proximal end to a distal end and which is capable of expanding or being expanded from a radially compressed state to a radially expanded state, characterised in that said tubular body is circumferentially reinforced along at least part of its length by a plurality of unit cells, wherein each unit cell comprises a first end portion comprising a plurality of tapering regions and a second end portion comprising at least one tapering region.

With reference to aspects five to eight, it is envisaged that the tubular body may comprise a sheath member which is reinforced by the unit cells. In this regard, the sheath member may be made from a biocompatible and flexible material such as Dacron™ or PTFE. The unit cells may be interwoven into the material of the sheath member or, alternatively, the unit cells may form a separate tubular structure analogous to that of the tubular body of the stent of aspects one to four wherein the sheath member substantially surrounds or is positioned within the tubular structure of unit cells.

Further description of the unit cells is understood to relate to both the stent and the graft as defined in the above aspects of the invention.

In one embodiment each unit cell is a multi-sided member. The multi-sided member preferably includes anywhere between six and fourteen sides and more preferably twelve sides. However, various number of sides and shapes of unit cells are envisaged.

Further, the unit cells of the stent and the graft may all have the same number of sides or, alternatively, a proportion of the unit cells may differ in number of sides from the remainder of unit cells. While the stent and the graft may have a plurality of unit cells of uniform size, it is also envisaged that a proportion of the unit cells of the stent or graft may be of a different size to the remainder of unit cells.

The sides of the unit cells may be relatively straight or may be curved or sinusoidal or any other suitable shape which may provide the unit cells with a certain amount of flexibility or spring-like properties. While only one side may be curved or sinusoidal as mentioned, it is also envisaged that a plurality or all sides of the unit cells have such a curved or sinusoidal shape. The advantage of providing unit cells with at last one side having a curved or sinusoidal shape is that, any length change during radial compression of the stent or the graft is compensated for by the spring-like properties of the unit cells.

In one embodiment, in at least some of the unit cells of the stent or the graft, one side may be omitted. It is envisaged that such an arrangement would provide a stent or a graft with a good degree of flexibility. Such a stent or graft may have particular application in the treatment of a curved portion of diseased vessel.

Preferably the first end portion of each unit cell comprises a plurality of tapering regions and preferably two tapering regions which terminate in two points at the first end. Further, the second end portion preferably comprises a single tapering region which terminates in a single point at the second end. In this embodiment, the first end portion is therefore of a greater diameter than the diameter of the second end portion.

Preferably, the unit cells are arranged in a circumferential series which extends at least partially around the circumference of the tubular body. More preferably, the circumferential series of unit cells extends around the entire circumference of the tubular body to form, in the case of the stent, a cylindrical tube of unit cells. With reference to the graft of the present invention, the unit cells may form a separate cylindrical tube which is overlaid with the sheath member or alternatively the cylindrical tube of unit cells or individual unit cells may be integrated or interwoven into the sheath member.

Desirably, at least one unit cell in the circumferential series is connected to or integral with an adjacent unit cell in said circumferential series. In the embodiment wherein the at least one unit cell is integral with an adjacent unit cell, said at least one unit cell and said adjacent unit cell preferably have at least one common side.

Where at least one unit cell of a circumferential series is connected to rather than integral with an adjacent unit cell, said at least one unit cell and adjacent unit cell are preferably connected by at least one strut member. The at least one strut member may be straight, curved or sinusoidal. Preferably, the at least one strut member is a zigzag or a V-shape.

While the at least one unit cell may be connected to the adjacent unit cell by one strut member, it is equally envisaged that the at least one unit cell and the adjacent unit cell are connected to each other by a plurality of strut members and preferably two strut members.

Typically, the entire length of the stent or graft is made up of or reinforced by, respectively, a plurality of circumferential series of unit cells. In this embodiment, at least some of the unit cells comprising one circumferential series may be longitudinally connected to or integral with corresponding unit cells of a second circumferential series.

In one embodiment wherein at least some unit cells of one circumferential series are integral with corresponding unit cells in another circumferential series, it is envisaged that at least part of the first end portion of one unit cell in one circumferential series and at least part of the second end portion of a corresponding unit cell in the other circumferential series have at least one common side and preferably two common sides.

In this regard, as discussed above, the first end portion of each unit cell may comprise two tapering regions each of which terminates in a point at the first end. Accordingly, in this embodiment, the two tapering regions together form an indent therebetween. The indent may be defined, therefore, by an inner wall of each of the tapering regions of the first end portion.

The inner walls of the tapering regions of the first end portion of one unit cell in a first circumferential series may be the walls which form the second end portion of a unit cell in a second circumferential series. Alternatively, the inner walls defining the indent of the first end portion of one unit cell in a first circumferential series may be the same walls which form one of the tapering regions of the first end portion of a unit cell of a second circumferential series.

In the embodiment of the invention where the unit cells between one circumferential series and another circumferential series are connected to each other rather than integral with each other, each circumferential series of unit cells may be arranged such that the first end or the first end portion of a unit cell of one circumferential series is longitudinally connected by at least one connector member to the second end or the second end portion of a unit cell of the second circumferential series. Alternatively, the first end or first end portion of a unit cell in one circumferential series may be longitudinally connected to the first end or the first end portion of a unit cell in a second circumferential series.

The at least one connector member may connect only one unit cell in one circumferential series with a second unit cell in another circumferential series. Alternatively, a plurality of unit cells of one circumferential series may be connected to a plurality of corresponding unit cells in another circumferential series by a connector member. All of the unit cells of one circumferential series may also be connected to a corresponding unit cell of another circumferential series.

The connector member may be straight but, equally, the connector member may be sinusoidal, curved, zig-zag shaped, V-shaped, substantially circular or oval or oblique relative to the longitudinal axis of the unit cells. More than one connector member may connect one unit cell of one circumferential series with a unit cell of another circumferential series.

In a further embodiment, the two tapering regions of the first end portion may be elongate in shape such that they overlap with the second end portion of a corresponding unit cell in another circumferential series. In this case, the unit cell having the elongate tapering regions may or may not be connected to the corresponding unit cell of the other circumferential series.

While it is envisaged that the unit cells of each circumferential series are circumferentially aligned around the tubular body, the unit cells may also be arranged in a staggered fashion around the circumference of the tubular body.

For example, typically, the unit cells of the stent or the graft are orientated such that the first end of each unit cell is positioned relatively closer to the proximal end of the tubular body than the second end of each unit cell. The unit cells of each circumferential series while still arranged in the same general orientation, may be staggered such that, for example, every second unit cell is closer to the proximal or, alternatively, to the distal end of the tubular body of the stent or the graft than its adjacent unit cell(s). It is also envisaged that every third, fourth, fifth, sixth etc unit cell could be staggered in this manner.

The unit cells may also form a circumferential spiral series or a number of circumferential spiral series around the tubular body of the stent or the graft.

As discussed above, in each circumferential series, the unit cells may vary in size or may be a uniform size. In one embodiment, every second unit cell of a circumferential series may be of a greater size than its adjacent unit cells such that said larger unit cell is adapted to span two or more circumferential series of unit cells.

The shape, size and configuration of unit cells may be formed during manufacture of a stent or a graft by laser cutting a tube of suitable material. In this regard, it is envisaged that a computer programmed arrangement and configuration of unit cells be loaded into the software of a laser cutter which is essentially a computer controlled indexing device which precisely rotates and longitudinally slides the tube of suitable material under a fixed laser beam. The laser beam cuts through the wall of the material of the tube as it is rotated and longitudinally moved.

Alternatively, the tubular body of the stent or the unit cells of the graft may be made of a continuous wire which may be subsequently shaped to form a suitable pattern of unit cells.

Suitable materials for extruding the unit cells include but are not limited to Nitinol™, stainless steel or other alloys such as tantalum or Elgiloy.

With reference to the stent of aspects one to four, the tubular body of unit cells may be formed from other suitable biocompatible materials, selected, for best results, on the basis of the material's capacity to withstand the compressive forces of the stenotic lesion and maintain patency of the vessel throughout the life of the stent.

Preferably, the cross-sectional diameter of the tubular body of the stent or the graft in its radially compressed state is less than 2 mm and in its radially expanded state more than 7 mm.

The stent of the present invention may be used to treat stenosis or other conditions of the visceral arteries such as the renal and mesenteric arteries, the iliac artery and the sub-clavian artery. It may also be used to treat stenotic lesions in the peripheral vasculature and the coronary circulation. However, the application of the invention for use in the treatment of stenotic disease is not to be understood as limited to the vascular system only. The stent may be used to treat stenotic lesions in other structures including, for example, those comprising the hepato-biliary and genito-urinary tracts.

The graft of the present invention may be used to treat aneurysmal disease of the arteries of a patient such as the aorta and including the renal and mesenteric arteries, the iliac artery and the sub-clavian artery. It may also be used to treat disease of the peripheral vasculature and the coronary circulation.

The stent or graft may be coated with any of a number of agents including but not limited to heparin, warfarin, ticloidine, dipyramole, GPIIb/IIIa receptor blockers, thromboxane inhibitors, seratonin antagonists, prostanoids, calcium channel blockers, ACE inhibitors, angiopeptin, steroids, non-steroidal anti-inflammatory drugs, enzymes, immune suppressants, chemotherapeutic agents, genetic modifiers and nitric oxide.

In a preferred embodiment, during use of the stent of the present invention, the tubular body is initially in the radially compressed state to enable delivery of the stent through an introducer catheter. Upon deployment of the stent into a selected vessel, the tubular body may be caused to expand, or may be allowed to self-expand into the expanded state.

The sheath member of the graft is also initially in the radially compressed state to enable delivery of the graft through an introducer catheter. If the unit cells form a separate tubular structure (rather than being interwoven into the sheath member), the tubular structure of unit cells is preferably packaged in a radially compressed configuration within the lumen of the sheath member or alternatively around said sheath member. Upon deployment of the graft into a selected vessel, the sheath member may be caused to expand, or may be allowed to self-expand into the expanded state.

There are at least three preferred mechanisms whereby the tubular body of the stent or the graft may change from the radially compressed state to the radially expanded state. For instance, the tubular body may be expanded by the force of an inflating balloon within said tubular body or by some other mechanically applied force.

Alternatively, the unit cells may be made from a shape memory material as mentioned above wherein the patient's body temperature causes the unit cells to take on a “memorised” shape.

In a further embodiment, the tubular body may be spring expandable following the release of the compressive force of an introducer catheter used to introduce the stent or graft into a target vessel.

In a ninth aspect, the invention relates to a method of positioning an intraluminal stent according to any one of the first to fourth aspects of the invention in a vessel of a patient, the method including the steps of:
(i) introducing a catheter or other delivery device into a vein, artery or other vessel in the body of a patient when the tubular body of the intraluminal stent is in its radially compressed state;
(ii) causing the intraluminal stent to be carried through the catheter or other delivery device to a target site of stenosis in a vessel;
(iii) causing or allowing the tubular body of the intraluminal stent to expand within the vessel; and
(iv) withdrawing the catheter or other delivery device along with any other apparatus used to introduce the intraluminal stent into the vessel from the body of the patient.

In a tenth aspect, the invention relates to a method of positioning an intraluminal graft according to any one of the fifth to eighth aspects of the invention in a vessel of a patient, the method including the steps of:
(i) introducing a catheter or other delivery device into a vein, artery or other vessel in the body of a patient when the tubular body of the intraluminal graft is in the radially compressed state;
(ii) causing the intraluminal graft to be carried through the catheter or other delivery device to a target site of stenosis in a vessel;
(iii) causing or allowing the tubular body of the intraluminal graft to expand within the vessel; and
(iv) withdrawing the catheter or other delivery device along with any other apparatus used to introduce the intraluminal graft into the vessel from the body of the patient.

In one embodiment, the stent or the graft may be pre-loaded with the catheter or other delivery device. Alternatively, the stent or the graft may be delivered to a target site as a separate step to the introduction of the catheter or other delivery device.

The stent or graft may have radio-opaque markers incorporated therein to enable a surgeon to view the position of the graft within the vessels. Alternatively, the material of the stent or graft may be radio-opaque.

### BRIEF DESCRIPTION OF THE DRAWINGS

FIG. 1 is a side elevational view of one embodiment of the stent of the present invention;

FIG. 2a is a depiction of a unit cell of one embodiment of the stent of the invention;

FIG. 2b is a side elevational view of another embodiment of the stent of the present invention;

FIGS. 3 to 12 depict various arrangements of unit cells of different embodiments of the stent of the present invention;

FIG. 13 is a depiction of a unit cell of a further embodiment of the stent of the present invention;

FIG. 14 is a side elevational view of one embodiment of the graft of the present invention;

FIG. 15a is a depiction of a unit cell of one embodiment of the graft of the invention;

FIG. 15b is a side elevational view of another embodiment of the graft of the present invention;

FIGS. 16 to 25 depict various arrangements of unit cells of different embodiments of the graft of the present invention;

FIG. 26 depicts a unit cell of a further embodiment of the graft of the present invention; and

FIG. 27 depicts a spiral arrangement of unit cells reinforcing the intraluminal graft.

### PREFERRED MODE OF CARRYING OUT THE INVENTION

The intraluminal stent of the present invention is generally depicted as 10 in the accompanying drawings. The intraluminal stent 10 comprises a tubular body 11 extending from a proximal end 12 to a distal end 13. The tubular body 11 includes a plurality of unit cells 14, each unit cell having a first end portion 15 adjacent a first end 16 and a second end portion 17 adjacent a second end 18. The first end portion 15 has a greater diameter than the diameter of the second end portion 17.

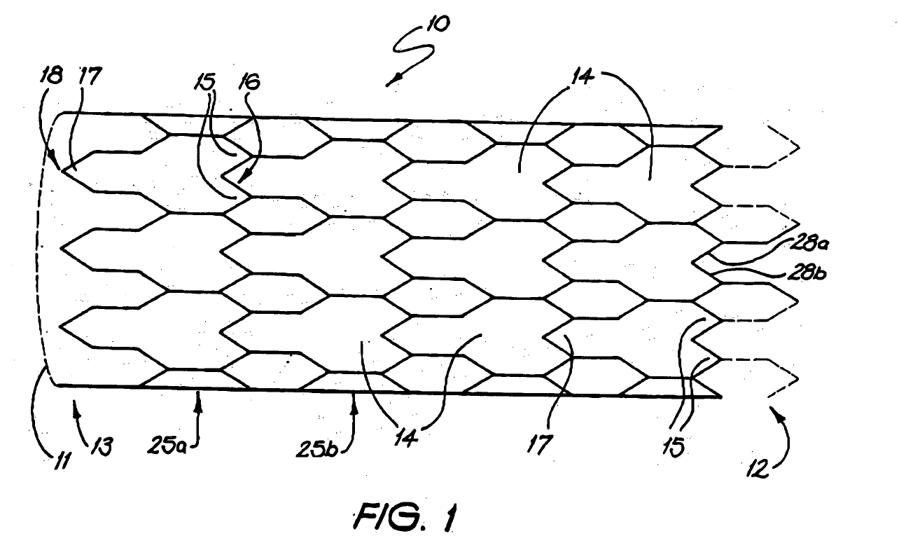

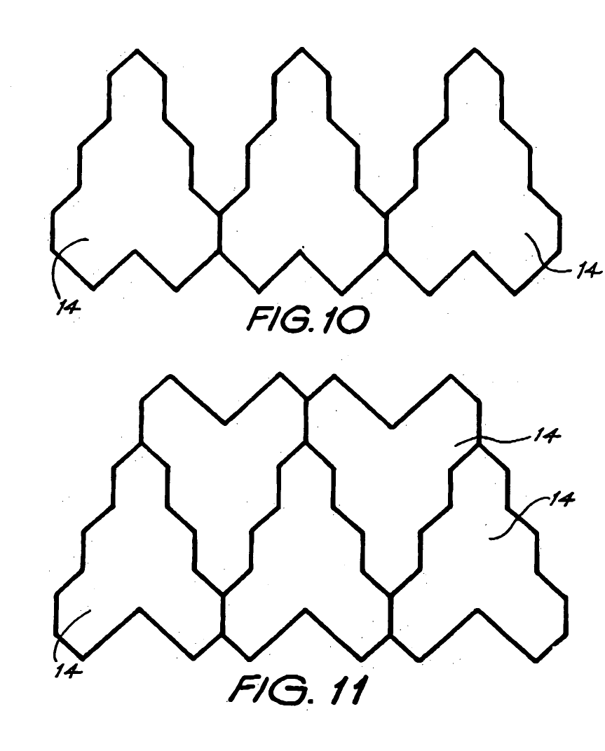

As depicted in the Figures, each unit cell is a multi-sided member. FIGS. 1 to 9 show a twelve sided unit cell 14 and FIGS. 10, 11 and 12 show a sixteen sided unit cell 14.

The first end portion 15 of a unit cell as shown in FIG. 1 comprises two tapering regions 21 which terminate in two points 22 at the first end 16. The second end portion 17 comprises a single tapering region 23 which terminates in a single point 24 at the second end 18.

FIG. 1 shows the tubular body 11 which is made up of a circumferential series of unit cells 14. The back wall of the tubular body 11 is not depicted in FIG. 1. The unit cells are arranged in a series 25 which extends around the circumference of the tubular body 11.

The unit cells 14 of FIG. 1 are integral with adjacent unit cells in the circumferential series 25 and each unit cell 14 has a common side 26 with an adjacent unit cell 14.

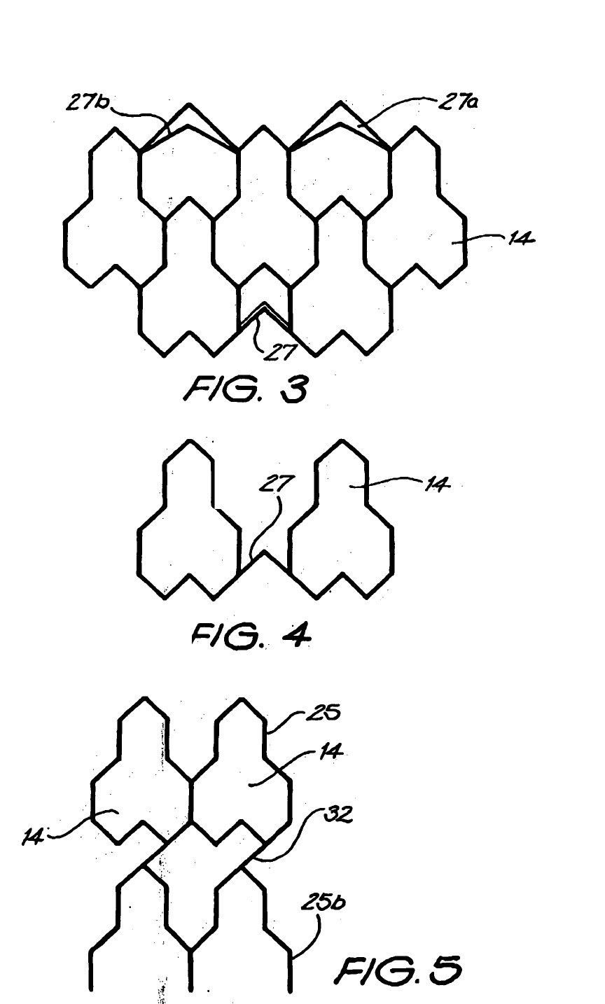

FIGS. 3 and 4 depict an arrangement wherein a unit cell 14 of a circumferentially arranged circumferential series 25 is connected to rather than integral with an adjacent unit cell 14. The connection is made by a strut member 27 or a number of strut members 27. In these Figures, the strut member 27 is shown as a V-shaped member connecting the unit members 14. In FIG. 3, it can be seen that two strut members 27a and 27b connect adjacent unit cells 14.

FIG. 1 shows that the entire length of the tubular body 11 is made up of a plurality of circumferential series 25 of unit cells 14. In this Figure, it can be seen that the unit cells of a first circumferential series 25a are integral with corresponding unit cells 14 of a second circumferential series 25b. Such an arrangement continues along the length of the tubular body 11.

At least part of the first end portion 15 of one unit cell 14 in the first circumferential series 25a and at least part of the second end portion 17 of a corresponding unit cell 14 in the second circumferential series 25b have two common sides 28a and 28b.

In this regard, as discussed above, the first end portion 15 of each unit cell 14 comprises two tapering regions 21 each of which terminates in a point 22 at the first end 16. The two points 22 together form an indent 29 defined by an inner wall 31 of each of the tapering regions 21 of the first end portion 15. The inner walls 31 of the tapering regions 21 of the first end portion 15 of one unit cell 14 in the first circumferential series 25a are shown in FIG. 1 to be the same walls which form the second end portion 17 of a unit cell 14 in the second circumferential series 25b.

FIG. 7 depicts an arrangement of unit cells 14 where the inner walls 31 defining the indent 29 of the first end portion 15 of one unit cell 14 in a first circumferential series 25a may be the same walls which form one of the tapering regions 21 of the first end portion 15 of a unit cell 14 of the second circumferential series 25b.

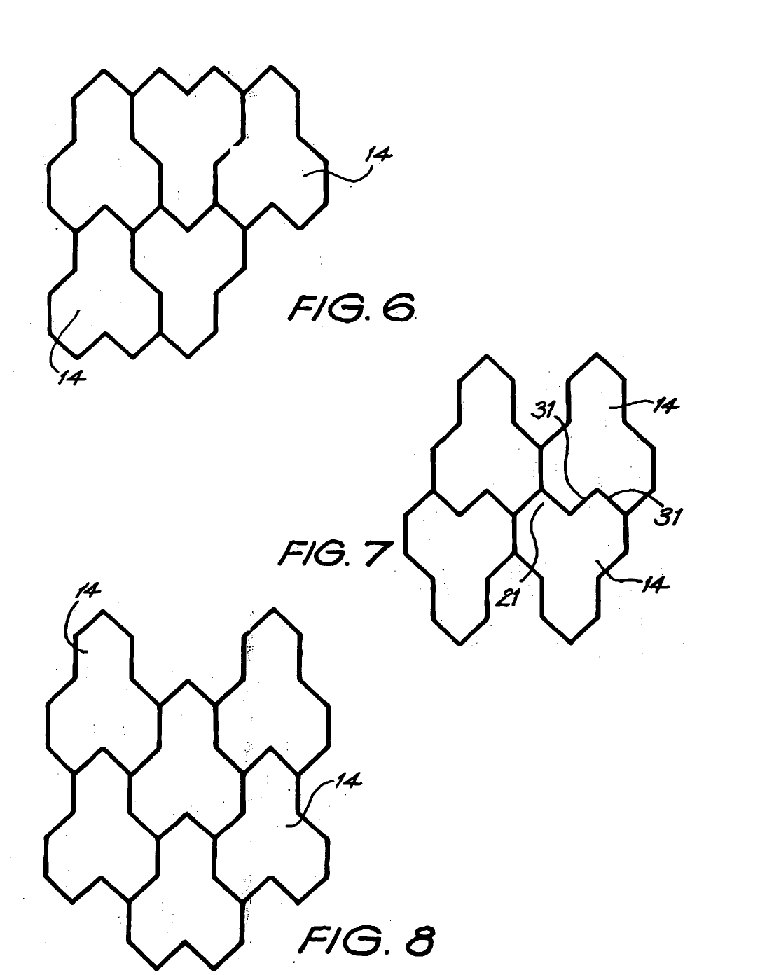

FIG. 6 shows an arrangement of unit cells 14 which combines both the arrangements of FIG. 1 and FIG. 7.

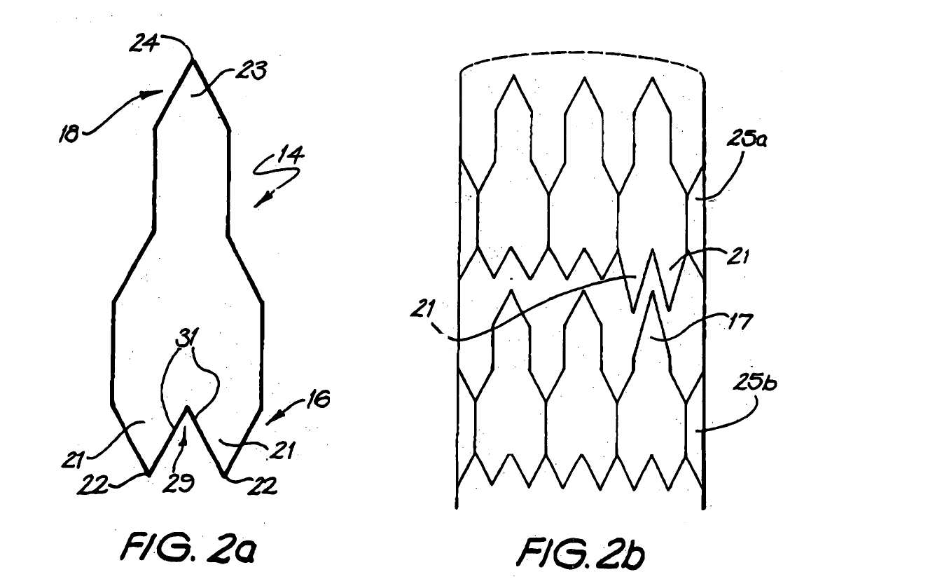

Rather than the above description wherein the unit cells 14 between the first circumferential series 25a and the second circumferential series 25b are integral with each other, FIG. 2b depicts an embodiment wherein the unit cells of each circumferential series 25 are not connected to the unit cells of another circumferential series 25.

FIG. 5 shows a further embodiment wherein the unit cells 14 of the first circumferential series 25a are connected to the unit cells 14 of the second circumferential series 25b by a connector 32.

In FIG. 2b, the two tapering regions 21 of the first end portion 15 of a unit cell 14 of the first circumferential series 25a are shown as more elongate in structure when compared to the other unit cells 14 of the first circumferential series 25a. The two tapering regions 21 overlap with the second end portion 17 of a corresponding unit cell 14 of the second circumferential series 25b.

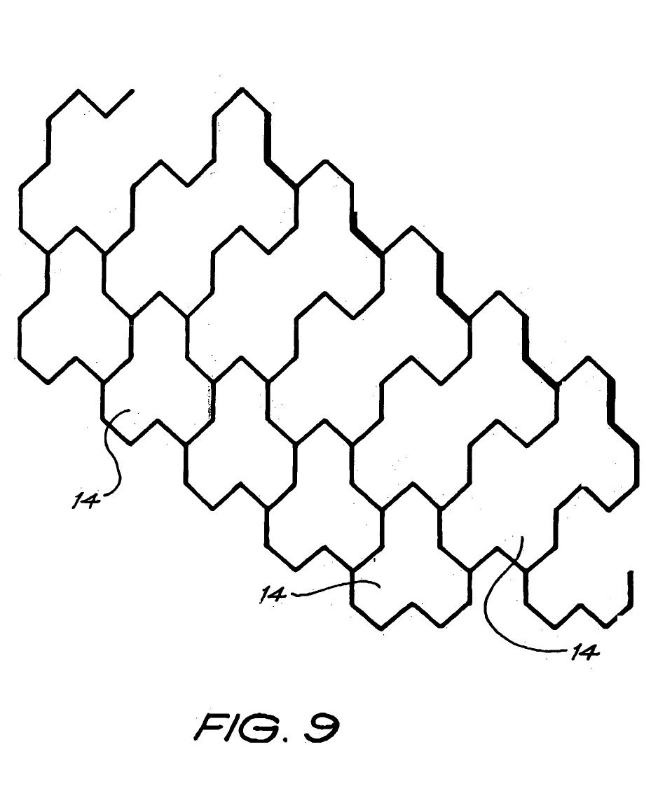

While the unit cells 14 of each circumferential series 25 may be circumferentially aligned on the tubular body 11 the unit cells 14, of each circumferentially arranged circumferential series 25 may be staggered in their arrangement as depicted in FIG. 8. As shown, every second unit cell 14 of a circumferential series 25 is staggered. Such staggering of the units cells 14 in each circumferential series 25 provides a spiral pattern of unit cells 14 around the circumference of the tubular body 11. This arrangement is generally depicted in FIG. 9.

While the unit cells 14 of the tubular body 11 may all be of the same shape having the same number of sides, a proportion of the unit cells 14 may differ from the remainder of unit cells 14 in shape, number of sides and size. A unit cell 14 of greater size than its adjacent unit cells 14 is depicted in FIG. 12. The larger unit cell 14 can be seen to span two circumferential series 25 of unit cells 14.

In FIG. 9, one side of the multi-sided unit cells 14 of the tubular body 11 is omitted in a proportion of the unit cells 14 of the tubular body 11. It is envisaged that such an arrangement would provide a stent having relatively good flexibility. Such a stent may have particular application in respect of a curved portion of vessel which requires stenting.

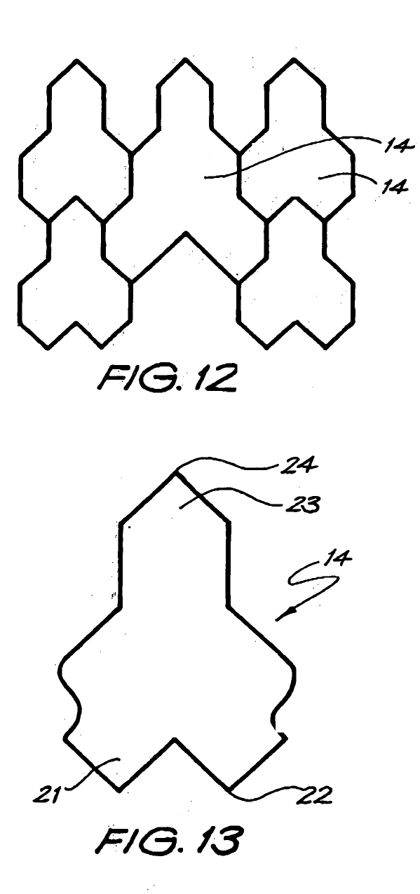

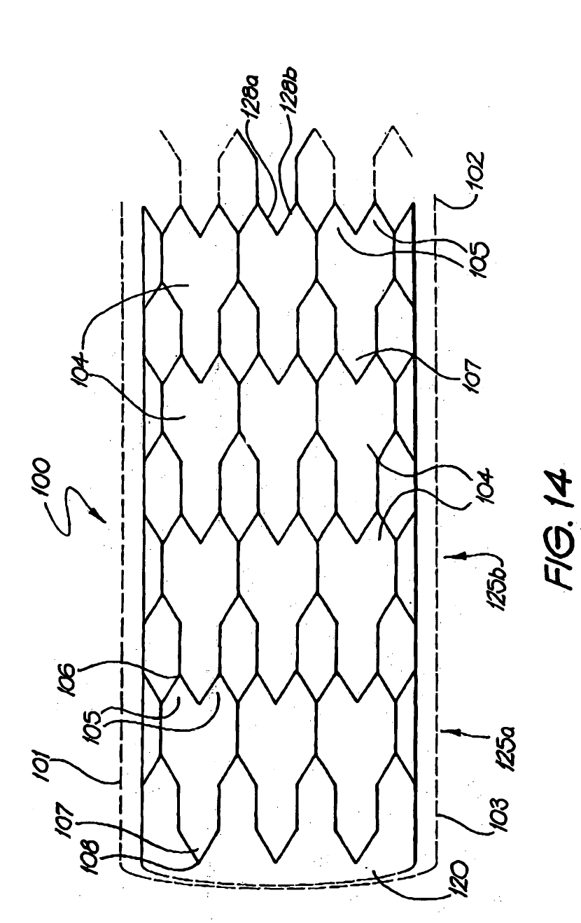

In FIG. 13, one side of a unit cell 14 is shown to be relatively sinusoidal in configuration. This provides the unit cell with a certain amount of flexibility or spring-like properties. The advantage of providing a unit cell with at last one side having a curved or sinusoidal shape is that, any length change during radial compression of the stent is compensated for by the spring-like properties of the stent.

The intraluminal graft of the present invention is generally depicted as 100 in the accompanying drawings. The intraluminal graft 100 comprises a tubular body 101 extending from a proximal end 102 to a distal end 103. The tubular body 101 is circumferentially reinforced by a plurality of unit cells 104, each unit cell having a first end portion 105 adjacent a first end 106 and a second end portion 107 adjacent a second end 108. The first end portion 105 has a greater diameter than the diameter of the second end portion 107.

The tubular body 101 may be circumferentially reinforced by the unit cells in a number of ways. For instance as depicted in FIG. 14, the unit cells may form an elongate cylinder 120 which is disposed within the lumen of the tubular body such that it acts as a scaffold for said tubular body 101. Alternatively, although not depicted, the unit cells may be interwoven within the structure of the tubular body thereby forming an integral scaffold. It is also envisaged that an elongate body 120 of unit cells 104 may surround the tubular body 101.

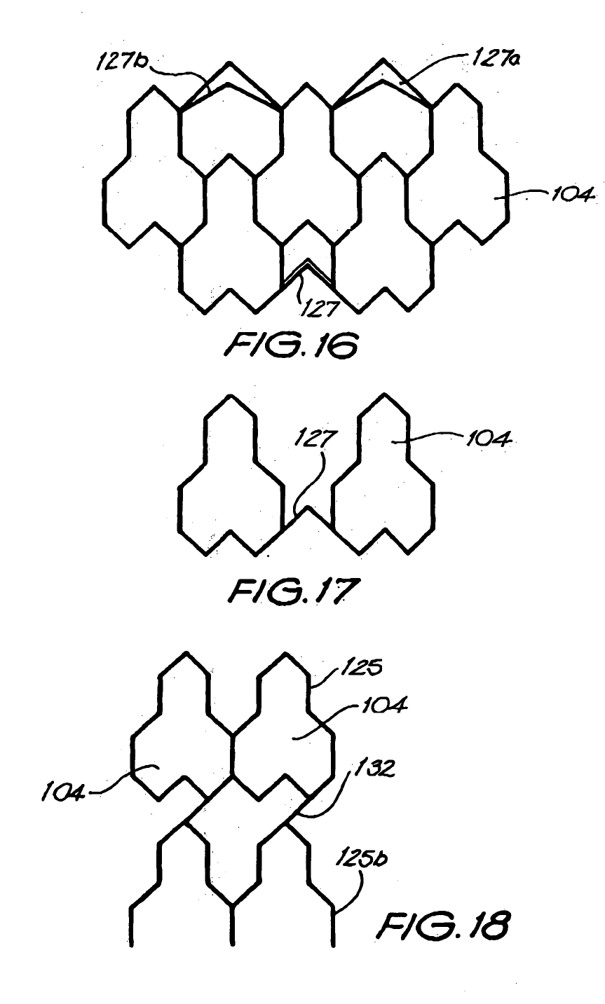

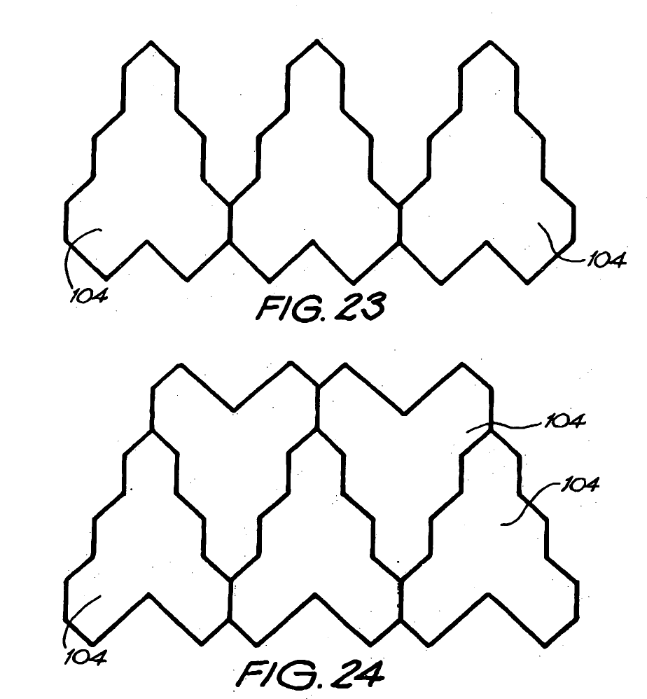

As depicted in the FIGS. 14 to 27, each unit cell 104 is a multi-sided member. FIGS. 14 to 22 and FIG. 27 show a twelve sided unit cell 104 and FIGS. 23, 24 and 25 show a sixteen sided unit cell 104.

The first end portion 105 of a unit cell as shown in FIG. 14 comprises two tapering regions 121 which terminate in two points 122 at the first end 106. The second end portion 107 comprises a single tapering region 123 which terminates in a single point 124 at the second end 108.

As depicted in FIG. 14, the unit cells 104 are arranged in a plurality of circumferential series 125 which together form an elongate cylinder 120.

The unit cells 104 of FIG. 14 are integral with adjacent unit cells in the circumferential series 125 and each unit cell 104 has a common side 126 with an adjacent unit cell 104.

FIGS. 16 and 17 depict an arrangement wherein a unit cell 104 of a circumferentially arranged circumferential series 125 is connected to rather than integral with an adjacent unit cell 104. The connection is made by a strut member 127 or a number of strut members 127. In these Figures, the strut member 127 is shown as a V-shaped member connecting the unit members 104. In FIG. 16, it can be seen that two strut members 127a and 127b connect adjacent unit cells 104.

FIG. 14 shows that the entire length of the tubular body 101 is reinforced by elongate cylinder 120. In this Figure, it can be seen that the unit cells of a first circumferential series 125a are integral with corresponding unit cells 104 of a second circumferential series 125b. Such an arrangement continues along the length of the tubular body 101.

At least part of the first end portion 105 of one unit cell 104 in the first circumferential series 125a and at least part of the second end portion 107 of a corresponding unit cell 104 in the second circumferential series 125b have two common sides 128a and 128b.

In this regard, as discussed above, the first end portion 105 of each unit cell 104 comprises two tapering regions 121 each of which terminates in a point 122 at the first end 106. The two points 122 together form an indent 129 defined by an inner wall 131 of each of the tapering regions 121 of the first end portion 105. The inner walls 131 of the tapering regions 121 of the first end portion 105 of one unit cell 104 in the first circumferential series 125a are shown in FIG. 14 to be the same walls which form the second end portion 107 of a unit cell 104 in the second circumferential series 125b.

FIG. 20 depicts an arrangement of unit cells 104 where the inner walls 131 defining the indent 129 of the first end portion 105 of one unit cell 104 in a first circumferential series 125a may be the same walls which form one of the tapering regions 121 of the first end portion 105 of a unit cell 104 of the second circumferential series 125b.

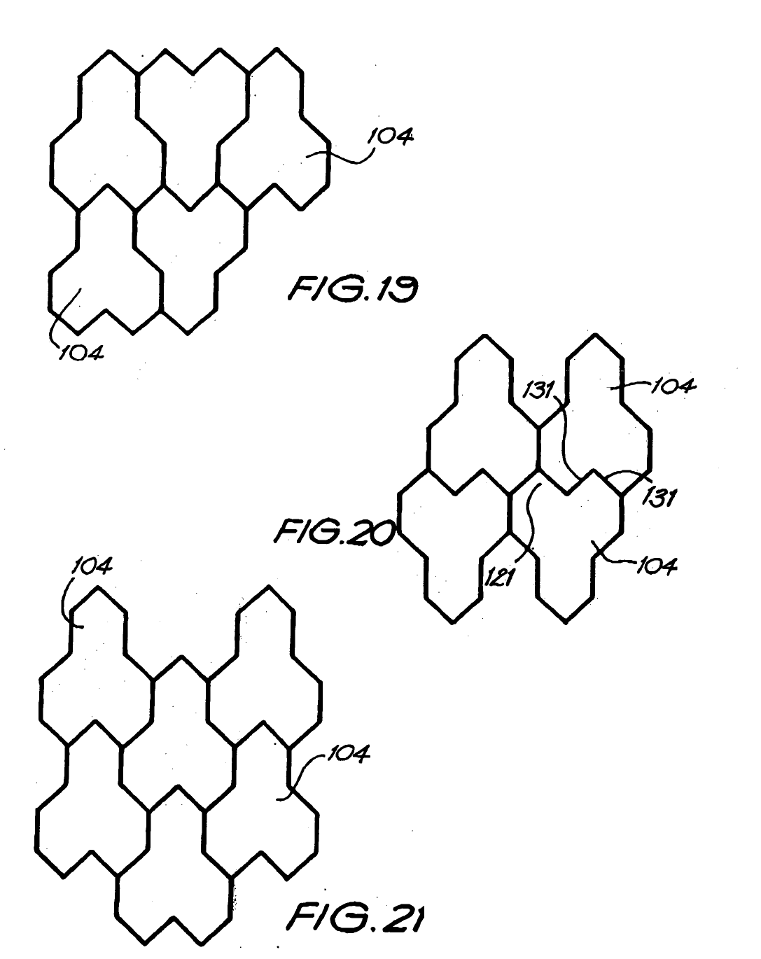

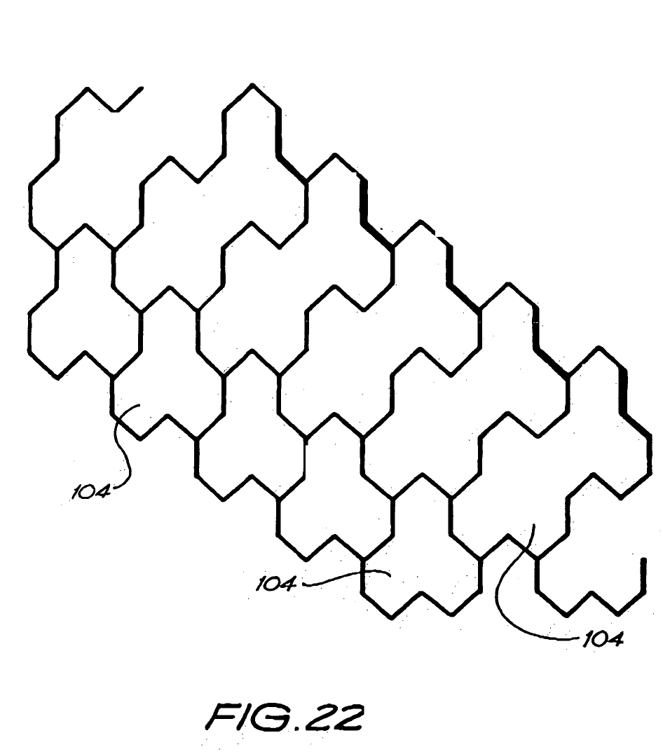

FIG. 20 shows an arrangement of unit cells 104 which combines both the arrangements of FIG. 14 and FIG. 21.

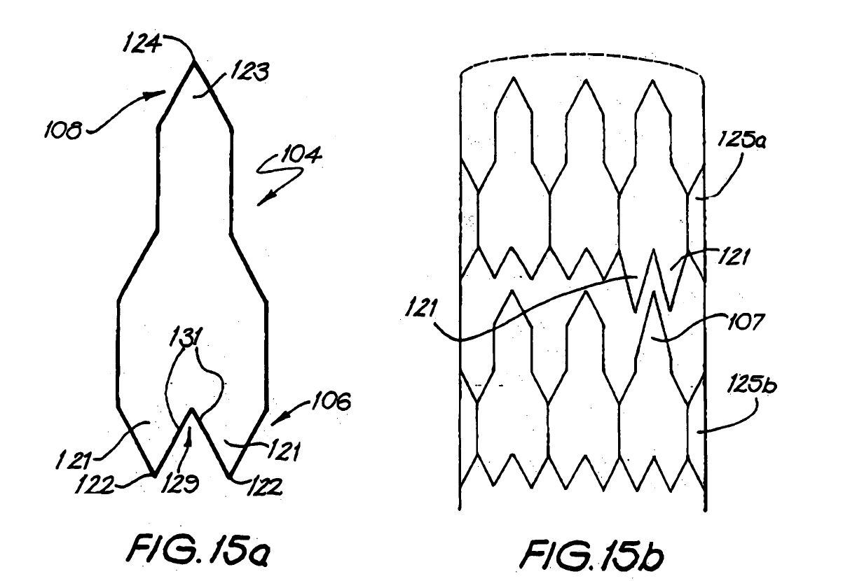

Rather than the above description wherein the unit cells 104 between the first circumferential series 125a and the second circumferential series 125b are integral with each other, FIG. 15b depicts an embodiment wherein the unit cells of each circumferential series 125 are not connected to the unit cells of another circumferential series 125.

FIG. 19 shows a further embodiment wherein the unit cells 104 of the first circumferential series 125a are connected to the unit cells 104 of the second circumferential series 125b by a connector 132.

In FIG. 15, the two tapering regions 121 of the first end portion 105 of a unit cell 104 of the first circumferential series 125a are shown as more elongate in structure when compared to the other unit cells 104 of the first circumferential series 125a. The two tapering regions 121 overlap with the second end portion 107 of a corresponding unit cell 104 of the second circumferential series 125b.

While the unit cells 104 of each circumferentially arranged circumferential series 125 may be circumferentially aligned on the tubular body 101 the unit cells 104, of each circumferentially arranged circumferential series 125 may be staggered in their arrangement as depicted in FIG. 21. As shown, every second unit cell 104 of a circumferential series 125 is staggered. This arrangement is generally depicted in FIG. 22.

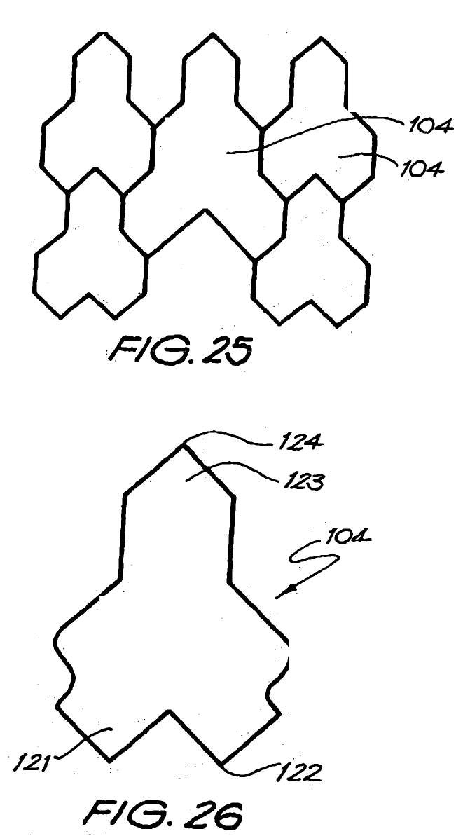

While the unit cells 104 of the intraluminal graft may all be of the same shape having the same number of sides, a proportion of the unit cells 104 may differ from the remainder of unit cells 104 in shape, number of sides and size. A unit cell 104 of greater size than its adjacent unit cells 104 is depicted in FIG. 25. The larger unit cell 104 can be seen to span two circumferential series 125 of unit cells 104.

In FIG. 22, one side of the multi-sided unit cells 104 of the intraluminal graft is omitted in a portion of the unit cells 104 of the graft. It is envisaged that such an arrangement would provide a graft having relatively good flexibility. Such a graft may have particular application in respect of a curved portion of vessel which requires grafting.

In FIG. 26, one side of a unit cell 104 is shown to be relatively sinusoidal in configuration. This provides the unit cell with a certain amount of flexibility of spring-like properties. The advantage of providing a unit cell with at last one side having a curved or sinusoidal shape is that, any length change during radial compression of the graft is compensated for by the spring-like properties of the unit cell 104.

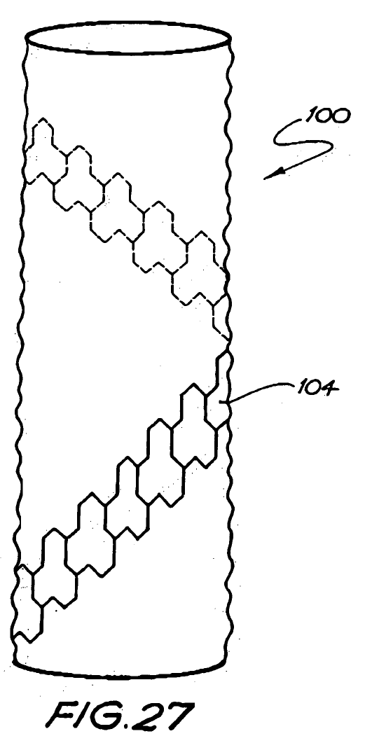

FIG. 27 depicts an embodiment of the invention wherein the unit cells 104 form a spiral series around the tubular body 101 of the graft 100. While a single spiral series is depicted, it is envisaged that a plurality of spiral series may be arranged around the tubular body.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An intraluminal stent comprising a tubular body extending from a proximal end to a distal end, said tubular body being capable of expanding or being expanded from a radially compressed state to a radially expanded state, characterised in that the tubular body includes a plurality of unit cells, each unit cell having a first end portion adjacent a first end and a second end portion adjacent a second end and wherein the first end portion is of a greater dimension than the dimension of the second end portion.

2. An intraluminal stent comprising a tubular body extending from a proximal end to a distal end, said tubular body being capable of expanding or being expanded from a radially compressed state to a radially expanded state, characterised in that the tubular body includes a plurality of unit cells, each unit cell having a longitudinal axis and a transverse axis, wherein each unit cell is symmetrical about its longitudinal axis and asymmetrical about its transverse axis.

3. An intraluminal stent comprising a tubular body extending from a proximal end to a distal end, said tubular body being capable of expanding or being expanded from a radially compressed state to a radially expanded state, characterised in that the tubular body includes a plurality of unit cells, each unit cell having a first end portion adjacent a first end and a second end portion adjacent a second end, wherein the first end portion is of a greater dimension than the dimension of the second end portion and each unit cell has a longitudinal axis and a transverse axis, each cell being symmetrical about its longitudinal axis and asymmetrical about its transverse axis.

4. An intraluminal stent comprising a tubular body extending from a proximal end to a distal end, said tubular body being capable of expanding or being expanded from a radially compressed state to a radially expanded state, characterised in that the tubular body includes a plurality of unit cells, wherein each unit cell comprises a first end portion comprising a plurality of tapering regions and a second end portion comprising at least one tapering region.

5. The intraluminal stent of any one of claims 1 to 4 wherein each unit cell is a multi-sided member.

6. The intraluminal stent of claim 5 wherein the each unit cell has between six and fourteen sides.

7. The intraluminal stent of claim 6 wherein each unit cell has twelve sides.

8. The intraluminal stent of any one of claims 1 and 3 to 7 wherein the first end portion of each unit cell comprises two tapering regions which terminate in two points at the first end.

9. The intraluminal stent of any one of claims 1 and 3 to 8 wherein the second end portion comprises a single tapering region which terminates in a single point at the second end.

10. The intraluminal stent of any one of the preceding claims wherein the unit cells are arranged in circumferential series which extend at least partially around the circumference of the tubular body.

11. The intraluminal stent of claim 10 wherein said circumferential series of unit cells extends around the entire circumference of the tubular body.

12. The intraluminal stent of claim 11 wherein at least one circumferential series is arranged in a spiral pattern around the tubular body of the intraluminal stent.

13. The intraluminal stent of any one of claims 10 to 12 wherein at least one unit cell in one or more circumferential series is connected to or integral with an adjacent unit cell in said one or more circumferential series.

14. The intraluminal stent of claim 13 wherein the at least one unit cell is integral with an adjacent unit cell and wherein said at least one unit cell and said adjacent unit cell have at least one common side.

15. The intraluminal stent of claim 12 wherein said at least one unit cell is connected to said adjacent unit cell by at least one strut member.

16. The intraluminal stent of any one of claims 10 to 15 wherein the entire length of the tubular body is made up of a plurality of circumferential series of unit cells and wherein at least some of the unit cells comprising one circumferential series are longitudinally connected to or integral with corresponding unit cells of a second circumferential series.

17. The intraluminal stent of claim 16 wherein at least part of the first end portion of at least one unit cell in said one circumferential series and at least part of the second end portion of a corresponding unit cell in said second circumferential series have at least one common side.

18. The intraluminal stent of claim 17 wherein the first end portion of said at least one unit cell of said one circumferential series comprises two tapering regions each of which terminates in a point at the first end and wherein an inner wall of each of said tapering regions together define an indented region, wherein further, the inner walls form the common side between said at least one unit cell of said one circumferential series and said corresponding unit cell of said second circumferential series.

19. The intraluminal stent of claim 18 wherein the inner walls of the two tapering regions of said one unit cell of said one circumferential series are the same walls which form the second end portion of the corresponding unit cell of the second circumferential series.

20. The intraluminal stent of claim 18 wherein the inner walls of the two tapering regions of said one unit cell of said one circumferential series are the same walls which form one of the tapering regions of the first end portion of the corresponding unit cell of the second circumferential series.

21. The intraluminal stent of claim 16 wherein said some of the unit cells comprising one circumferential series are longitudinally connected to corresponding unit cells of a second circumferential series by at least one connector member

22. The intraluminal stent of claim 21 wherein the connector member is sinusoidal, curved, zig-zag shaped, V-shaped, substantially circular or oval or oblique relative to the longitudinal axis of the unit cells.

23. The intraluminal stent of any one of the preceding claims wherein the tubular body is made from a material including Nitinol™, stainless steel or other alloys including tantalum or Elgiloy.

24. The intraluminal stent of any one of the preceding claims when used to treat stenosis or other conditions of the visceral arteries such as the renal and mesenteric arteries, the iliac artery and the sub-clavian artery and stenotic lesions in the peripheral vasculature, the coronary circulation, the hepato-biliary and genito-urinary tracts.

25. The intraluminal stent of any one of the preceding claims wherein the tubular body is coated with an agent including heparin, warfarin, ticloidine, dipyramole, GPIIb/IIIa receptor blockers, thromboxane inhibitors, seratonin antagonists, prostanoids, calcium channel blockers, ACE inhibitors, angiopeptin, steroids, non-steroidal anti-inflammatory drugs, enzymes, immune suppressants, chemotherapeutic agents, genetic modifiers and nitric oxide.

26. A method of positioning an intraluminal stent according to any one of the preceding claims in a vessel of a patient, the method including the steps of:
(i) introducing a catheter or other delivery device into a vein, artery or other vessel in the body of a patient when the tubular body of the intraluminal stent is in its radially compressed state;
(ii) causing the intraluminal stent to be carried through the catheter or other delivery device to a target site of stenosis in a vessel;
(iii) causing or allowing the tubular body of the intraluminal stent to expand within the vessel; and
(iv) withdrawing the catheter or other delivery device along with any other apparatus used to introduce the intraluminal stent into the vessel from the body of the patient.

27. An intraluminal graft comprising a tubular body which extends from a proximal end to a distal end and which is capable of expanding or being expanded from a radially compressed state to a radially expanded state, characterised in that said tubular body is circumferentially reinforced along at least part of its length by a plurality of unit cells, each unit cell having a first end portion adjacent a first end and a second end portion adjacent a second end, wherein the first end portion is of a greater dimension than the dimension of the second end portion.

28. An intraluminal graft comprising a tubular body which extends from a proximal end to a distal end and which is capable of expanding or being expanded from a radially compressed state to a radially expanded state, characterised in that said tubular body is circumferentially reinforced along at least part of its length by a plurality of unit cells, each unit cell having a longitudinal axis and a transverse axis, wherein each unit cell is symmetrical about its longitudinal axis and asymmetrical about its transverse axis.

29. An intraluminal graft comprising a tubular body which extends from a proximal end to a distal end and which is capable of expanding or being expanded from a radially compressed state to a radially expanded state, characterised in that said tubular body is circumferentially reinforced along at least part of its length by a plurality of unit cells, each unit cell having a first end portion adjacent a first end and a second end portion adjacent a second end, wherein the first end portion is of a greater dimension than the dimension of the second end portion and each unit cell has a longitudinal axis and a transverse axis, each cell being symmetrical about its longitudinal axis and asymmetrical about its transverse axis

30. An intraluminal graft comprising a tubular body which extends from a proximal end to a distal end and which is capable of expanding or being expanded from a radially compressed state to a radially expanded state, characterised in that said tubular body is circumferentially reinforced along at least part of its length by a plurality of unit cells, wherein each unit cell comprises a first end portion comprising a plurality of tapering regions and a second end portion comprising at least one tapering region.

31. The intraluminal graft of any one of claims 27 to 30 wherein the tubular body comprises a sheath member which is reinforced by the unit cells.

32. The intraluminal graft of claim 31 wherein the sheath member is made from a biocompatible and flexible material including Dacron™ or PTFE.

33. The intraluminal graft of claim 31 or claim 32 wherein the unit cells are interwoven into the material of the sheath member.

34. The intraluminal graft of claim 31 or claim 32 wherein the unit cells form a separate tubular structure to that of the sheath member and wherein the sheath member substantially surrounds or is positioned internal the tubular structure of unit cells.

35. The intraluminal graft of any one of claims 27 to 34 wherein each unit cell is a multi-sided member.

36. The intraluminal graft of claim 35 wherein the each unit cell has between six and fourteen sides.

37. The intraluminal graft of claim 36 wherein each unit cell has twelve sides.

38. The intraluminal graft of any one of claims 27 and 29 to 37 wherein the first end portion of each unit cell comprises two tapering regions which terminate in two points at the first end.

39. The intraluminal graft of any one of claims 27 and 29 to 38 wherein the second end portion comprises a single tapering region which terminates in a single point at the second end.

40. The intraluminal graft of any one of claims 27 to 39 wherein the unit cells are arranged in circumferential series which extend at least partially around the circumference of the tubular body.

41. The intraluminal graft of claim 40 wherein said circumferential series of unit cells extends around the entire circumference of the tubular body.

42. The intraluminal graft of claim 41 wherein at least one circumferential series is arranged in a spiral pattern around the tubular body of the intraluminal graft.

43. The intraluminal graft of any one of claims 40 to 42 wherein at least one unit cell in one or more circumferential series is connected to or integral with an adjacent unit cell in said one or more circumferential series.

44. The intraluminal graft of claim 43 wherein the at least one unit cell is integral with an adjacent unit cell and wherein said at least one unit cell and said adjacent unit cell have at least one common side.

45. The intraluminal graft of claim 44 wherein said at least one unit cell is connected to said adjacent unit cell by at least one strut member.

46. The intraluminal graft of any one of claims 40 to 45 wherein the entire length of the tubular body is made up of a plurality of circumferential series of unit cells and wherein at least some of the unit cells comprising one circumferential series are longitudinally connected to or integral with corresponding unit cells of a second circumferential series.

47. The intraluminal graft of claim 46 wherein at least part of the first end portion of at least one unit cells in said one circumferential series and at least part of the second end portion of a corresponding unit cell in said second circumferential series have at least one common side.

48. The intraluminal graft of claim 47 wherein the first end portion of said at least one unit cell of said one circumferential series comprises two tapering regions each of which terminates in a point at the first end and wherein an inner wall of each of said tapering regions together define an indented region, wherein further, the inner walls form the common side between said at least one unit cell of said one circumferential series and said corresponding unit cell of said second circumferential series.

49. The intraluminal graft of claim 48 wherein the inner walls of the two tapering regions of said one unit cell of said one circumferential series are the same walls which form the second end portion of the corresponding unit cell of the second circumferential series.

50. The intraluminal graft of claim 48 wherein the inner walls of the two tapering regions of said one unit cell of said one circumferential series are the same walls which form one of the tapering regions of the first end portion of the corresponding unit cell of the second circumferential series.

51. The intraluminal graft of claim 46 wherein said some of the unit cells comprising one circumferential series are longitudinally connected to corresponding unit cells of a second circumferential series by at least one connector member

52. The intraluminal graft of claim 51 wherein the connector member is sinusoidal, curved, zig-zag shaped, V-shaped, substantially circular or oval or oblique relative to the longitudinal axis of the unit cells.

53. The intraluminal graft of any one of claims 27 to 52 wherein the tubular body is made from a material including Nitinol™, stainless steel or other alloys including tantalum or Elgiloy.

54. The intraluminal graft of any one of claims 27 to 53 when used to treat aneurysmal disease of the arteries of a patient including the aorta, renal and mesenteric arteries, the iliac artery, the sub-clavian artery and diseases of the peripheral vasculature and the coronary circulation.

55. The intraluminal graft of any one of claims 27 to 54 wherein the tubular body is coated with an agent including heparin, warfarin, ticloidine, dipyramole, GPIIb/IIIa receptor blockers, thromboxane inhibitors, seratonin antagonists, prostanoids, calcium channel blockers, ACE inhibitors, angiopeptin, steroids, non-steroidal anti-inflammatory drugs, enzymes, immune suppressants, chemotherapeutic agents, genetic modifiers and nitric oxide.

56. A method of positioning an intraluminal graft according to any one of claims 27 to 55 in a vessel of a patient, the method including the steps of:
(i) introducing a catheter or other delivery device into a vein, artery or other vessel in the body of a patient when the tubular body of the intraluminal graft is in the radially compressed state;
(ii) causing the intraluminal graft to be carried through the catheter or other delivery device to a target site of stenosis in a vessel;
(iii) causing or allowing the tubular body of the intraluminal graft to expand within the vessel; and
(iv) withdrawing the catheter or other delivery device along with any other apparatus used to introduce the intraluminal graft into the vessel from the body of the patient.

57. An intraluminal stent substantially as hereinbefore described with reference to accompanying FIGS. 1 to 13.

58. An intraluminal graft substantially as hereinbefore described with reference to FIGS. 14 to 27.
